SEQUENCE LISTING (17 sequences)

SEQ ID NO: 1 - DNA, 20 bp, Artificial Sequence
  primer
gtcttaagcaattaagatag

SEQ ID NO: 2 - DNA, 24 bp, Artificial Sequence
  primer
cgcgtaagtaacctacctctaagt

SEQ ID NO: 3 - DNA, 18 bp, Artificial Sequence
  primer
gcttcgggaaactgaagg

SEQ ID NO: 4 - DNA, 20 bp, Artificial Sequence
  primer
tggrccgacatatgttggtt

SEQ ID NO: 5 - DNA, 20 bp, Artificial Sequence
  primer
cactaaagccgtaaggcgct

SEQ ID NO: 6 - DNA, 20 bp, Artificial Sequence
  primer
tggtgaggggcttgcgtccg

SEQ ID NO: 7 - DNA, 20 bp, Artificial Sequence
  primer
gtgagcgtaggtggtctttc

SEQ ID NO: 8 - DNA, 20 bp, Artificial Sequence
  primer
cagcaggagaaaacggaatt

SEQ ID NO: 9 - DNA, 19 bp, Artificial Sequence
  primer
gtatagggagtatcgaccc

SEQ ID NO: 10 - DNA, 25 bp, Artificial Sequence
  primer
tgtagtagtgaactgaaaggggaac

SEQ ID NO: 11 - DNA, 25 bp, Artificial Sequence
  primer
gacctgttaagtcaggaacttgcac

SEQ ID NO: 12 - DNA, 19 bp, Artificial Sequence
  primer
tgttgctagttaaattttc

SEQ ID NO: 13 - DNA, 20 bp, Artificial Sequence
  primer
gttgcaacagtgcgaactgg

SEQ ID NO: 14 - DNA, 19 bp, Artificial Sequence
  primer
gctaatccccaaagctgtc

SEQ ID NO: 15 - DNA, 21 bp, Artificial Sequence
  primer
gtcgatgtgccaaccgcaagg

SEQ ID NO: 16 - DNA, 21 bp, Artificial Sequence
  primer
acggytaccttgttaggactt

SEQ ID NO: 17 - DNA, 17 bp, Artificial Sequence
  primer
gtttgatcctggctcag

(19) United States
(12) Patent Application Publication
(10) Pub. No.: US 2003/0104383 A1
(43) Pub. Date: Jun. 5, 2003

(54) Nucleic acid, nucleic acid for detecting chlorinated ethylene-decomposing bacteria, probe, method of detecting chlorinated ethylene-decomposing bacteria, and method of decomposing chlorinated ethylene or ethane

(76) Inventors: Kanji Nakamura, Atsugi-Shi (JP); Toshihiro Ueno, Atsugi-Shi (JP)

Correspondence Address: Darby & Darby P.C., P.O. Box 5257, New York, NY, 10150-5257, US

(21) Appl. No.: 09/911,860

(22) Filed: Jul. 24, 2001

(30) Foreign Application Priority Data
Jul. 24, 2000 (JP) 2000-227580
Mar. 9, 2001 (JP) 2001-066001

(51) Int. Cl. C12Q 1/68; C12S 1/00; B09B 3/00; C12N 1/21

(52) U.S. Cl. 435/6; 435/252.3; 435/262.5

(57) ABSTRACT

Chlorinated ethylene-decomposition bacteria is detected by performing PCR using nucleic acid comprising 18˜25 nucleotides that preferentially hybridizes to the 16S rRNA or rDNA of chlorinated ethylene-decomposing bacteria and has any of base sequences of SEQ ID No. 1˜15, a base sequence that has at least 90% homology with any of these base sequences, or a base sequence complementary to any of these base sequences as the primer and the nucleic acid in a sample as the template. The DNA fragment that has been synthesized is detected. Chlorinated ethylene or ethane is decomposed by introducing the chlorinated ethylene-decomposing bacteria detected by this method to contaminated soil or underground water.

### TECHNICAL FIELD OF THE INVENTION

The present invention pertains to nucleic acid that preferentially hybridizes to the 16S rRNA or rDNA of chlorinated ethylene-decomposing bacteria. The present invention further pertains to a labeled probe for detecting chlorinated ethylene-decomposing bacteria comprising this nucleic acid, and a method of detecting chlorinated ethylene-decomposing bacteria using this nucleic acid or labeled probe. Additionally, the present invention pertains to a method of decomposing chlorinated ethylene or ethane.

### BACKGROUND OF THE INVENTION

A conventional method of purifying soil, underground water, or the like, contaminated by chlorinated ethylene or ethane, is a method of anaerobic dechlorination of chlorinated ethylene using the chlorinated ethylene-decomposing bacteria that are naturally present in contaminated soil. Moreover, methods of adding these bacteria to contaminated soil or underground water are also known. It is also a known fact that chlorinated ethylene-decomposing bacteria are capable of decomposing not only chlorinated ethylene, but also chlorinated ethane, using the chlorinated ethylene-decomposing enzymes that they possess. However, there are problems with this type of method in that very good treatment results, that is, thorough dechlorination, are not guaranteed.

Therefore, there is a demand for a method of pre-determining whether or not dechlorination can be accomplished when soil, underground water, or the like, contaminated by chlorinated ethylene or ethane, is to be purified using chlorinated ethylene-decomposing bacteria.

### OBJECTS AND SUMMARY OF THE INVENTION

It is an object of the present invention to present novel and useful nucleic acid that can be used for detection of chlorinated ethylene-decomposing bacteria. More specifically, the present invention provides nucleic acid that preferentially hybridizes to the 16S rRNA or rDNA of chlorinated ethylene-decomposing bacteria, a labeled probe for detection of chlorinated ethylene-decomposing bacteria comprising this nucleic acid, and a method of detecting chlorinated ethylene-decomposing bacteria using this nucleic acid or labeled probe and a method of decomposing chlorinated ethylene or ethane.

Briefly stated, the present invention relates to the following nucleic acid that preferentially hybridizes to the 16S rRNA or rDNA of chlorinated ethylene-decomposing bacteria, a labeled probe for detecting chlorinated ethylene-decomposing bacteria comprising this nucleic acid, a method of detecting chlorinated ethylene-decomposing bacteria using this nucleic acid or labeled probe, and method of decomposing chlorinated ethylene or ethane.

The present invention includes the following:

(1) Nucleic acid comprising 18˜25 nucleotides, which preferentially hybridizes to the 16S rRNA or rDNA of chlorinated ethylene-decomposing bacteria and has any of base sequence of SEQ ID No. 1 through No. 15, a base sequence having at least 90% homology with these base sequences, or a base sequence complementary to these base sequences.

(2) Nucleic acid comprising 10˜50 nucleotides that preferentially hybridize to the 16S rRNA or rDNA of chlorinated ethylene-decomposing bacteria wherein the base sequence of at least 10 individual bases in succession is the same as any of base sequences of SEQ ID No. 1 through No. 15 or complementary to these sequences.

(3) The use of the nucleic acid in above-mentioned (1) or (2) for the detection of chlorinated ethylene-decomposing bacteria.

(4) A labeled probe for the detection of chlorinated ethylene-decomposing bacteria, comprising nucleic acid in any of above-mentioned (1) through (3) which is labeled by a radioactive element, enzyme, fluorescent substance, antigen, antibody, or chemical substance.

(5) A method of detecting chlorinated ethylene-decomposing bacteria, comprising performing PCR (polymerase chain reaction) using the nucleic acid in any of above-mentioned (1) through (3) as the primer and the nucleic acid in a sample as the template, and detecting the DNA fragment that has been synthesized.

(6) A method of detecting chlorinated ethylene-decomposing bacteria, comprising bringing the labeled probe for detecting chlorinated ethylene-decomposing bacteria in above-mentioned (4) into contact with a sample or nucleic acid prepared from a sample to perform RNA or DNA hybridization, and detecting chlorinated ethylene-decomposing bacteria using the label as the indicator.

(7) A method of decomposing chlorinated ethylene or ethane, comprising performing the detection of chlorinated ethylene-decomposing bacteria in above-mentioned (5) or (6) using underground water or soil as the sample, and introducing the underground water or soil, in which chlorinated ethylene-decomposing bacteria have been detected, or cultivation liquid inoculated with these, to soil or underground water contaminated by chlorinated ethylene or ethane.

The above, and other objects, features and advantages of the present invention will become apparent from the following description read in conjunction with the accompanying drawings, in which like reference numerals designate the same elements.

### BRIEF DESCRIPTION OF THE DRAWINGS

FIG. 1 is a graph showing the results of Example 3.

FIG. 2 is a graph showing the results of the control in Example 3.

FIG. 3 is a graph showing the results of Example 4.

### DETAILED DESCRIPTION OF THE INVENTION

As a result of studying the reason why treatment results are always not very good with the conventional method of purification of soil, underground water, or the like, contaminated by chlorinated ethylene or ethane, using chlorinated ethylene-decomposing bacteria, the inventors clarified the fact that treatment results are good when chlorinated ethylene-decomposing bacteria, that perform dechlorination, live at the treated site. Treatment results cannot be expected when chlorinated ethylene-decomposing bacteria do not live at the treated site. Consequently, it is possible to judge whether or not treatment is thorough by examining the soil and underground water of the subject site and confirming whether or not chlorinated ethylene-decomposing bacteria live at that site.

It is possible to detect chlorinated ethylene-decomposing bacteria and thereby make the above-mentioned judgment by using the nucleic acid of the present invention.

The nucleic acid of the present invention is nucleic acid, comprising 18˜25 nucleotides, which preferentially hybridizes to the 16S rRNA or rDNA of chlorinated ethylene-decomposing bacteria and has any of base sequences of SEQ ID No. 1 through No.15 of the base sequence table. The nucleic acid of the present invention may have a base sequence having at least 90% homology with these base sequences, or a base sequence complementary to these base sequences.

Moreover, the nucleic acid of the present invention is nucleic acid, comprising 10˜50 nucleotides, preferably 15˜35 nucleotides, that preferentially hybridizes to the 16S rRNA or rDNA of chlorinated ethylene-decomposing bacteria. The base sequence of at least 10 individual bases in succession is the same as any of sequences of SEQ ID No. 1 through 15 or complementary to these base sequences. An example is nucleic acid having the same base sequence as a base sequence of 10 or more individual bases in succession beginning at any position in base sequence of SEQ ID No. 1. A base may be bound upstream and/or downstream of the base sequence that is the same as this base sequence of SEQ ID No. 1.

The nucleic acid of the present invention, that is, any of base sequences of SEQ ID No. 1 through 15, a base sequence having at least 90% homology with any of these base sequences, a base sequence complimentary to any of these base sequences, or a base sequence wherein the base sequence of at least 10 individual bases in succession is the same as any of base sequence of SEQ ID No. 1 through No. 15, or complementary to these base sequences, is easily chemically synthesized by conventional methods.

The base sequence of the 16S rDNA of chlorinated ethylene-decomposing bacteria has been determined. The nucleic acids of the present invention are designed using the specific segment of these sequences and therefore, they preferentially hybridize to 16S rRNA or rDNA of chlorinated ethylene-decomposing bacteria.

A specific example of the above-mentioned chlorinated ethylene-decomposing bacteria include bacteria belonging to the genus Dehalococcoides.

Specific examples of chlorinated ethylene that are decomposed (dechlorinated) by chlorinated ethylene-decomposing bacteria are tetrachloroethylene, trichloroethylene (TCE), cis-1,2-dichloroethylene, trans-1,2-dichloroethylene, 1,1-dichloroethylene, vinyl chloride, and their dechlorination intermediates. Moreover, specific examples of chlorinated ethanes that can be decomposed (dechlorinated) by chlorinated ethylene-decomposing bacteria are 1,2-dichloroethane, monochloroethane.

As will be mentioned later, chlorinated ethylene-decomposing bacteria can be detected easily at specifically high reliability by PCR using the nucleic acids of the present invention as the primer or by hybridization.

The labeled probe for detection of chlorinated ethylene-decomposing bacteria of the present invention is a probe wherein the above-mentioned nucleic acid of the present invention has been labeled with a label, such as a radioactive element, fluorescent substance, chemical substance, antigen, antibody, enzyme, or the like. Conventional labels can be used as this label, specific examples being radioactive elements, such as ³²P; fluorescent substances, such as FITC (fluorescence isothiocyanate) and rhodamine; haptenes, such as digoxygenin; enzymes, such as alkaline phosphatase and peroxidase; and chemical substances, such as biotin. These labels can be introduced to the nucleic acid by conventional methods.

The labeled probe for detecting chlorinated ethylene-decomposing bacteria of the present invention hybridizes with the sample to be checked for the presence of chlorinated ethylene-decomposing bacteria. The chlorinated ethylene-decomposing bacteria that have hybridized with labeled probe can be detected easily with specifically high reliability using this label as the indicator.

The method of detecting chlorinated ethylene-decomposing bacteria of the present invention is a method of detecting chlorinated ethylene-decomposing bacteria using the above-mentioned nucleic acid of the present invention. That is, PCR is performed using the above-mentioned nucleic acid of the present invention as the primer and the nucleic acid prepared from the sample to be checked for the presence of chlorinated ethylene-decomposing bacteria as the template. If DNA of the expected size is synthesized, it can be concluded that chlorinated ethylene-decomposing bacteria are present in the sample.

PCR can be performed by conventional methods, or it can be performed using a commercial PCR kit. PCR usually uses 2 types of primers, an upper primer and a lower primer, but the nucleic acid of the present invention can be used as one or both primers. Detection reliability can be improved by performing detection several times using several different types of nucleic acids as the primer.

Moreover, the method of detecting chlorinated ethylene-decomposing bacteria of the present invention is the method wherein chlorinated ethylene-decomposing bacteria are detected using the above-mentioned labeled probe for detection of chlorinated ethylene-decomposing bacteria of the present invention. That is, it is the method wherein, once RNA or DNA hybridization has been performed by bringing the above-mentioned labeled probe for detecting chlorinated ethylene-decomposing bacteria of the present invention into contact with the sample to be checked for presence of chlorinated ethylene-decomposing bacteria or with nucleic acid prepared from this sample, chlorinated ethylene-decomposing bacteria are detected using the label as the indicator. Hybridization can be performed by the same methods as conventional methods.

Detection after hybridization can be performed by conventional methods in accordance with the type of label. For instance, detection can be performed by assaying radioactivity by conventional methods when the probe has been labeled by a radioactive element. Moreover, detection can be performed by measuring the quantity of light by conventional methods when the probe has been labeled by a fluorescent substance. In addition, detection can be performed by assaying enzyme activity by conventional methods when the probe has been labeled by an enzyme. Furthermore, detection can be performed by conducting an antigen-antibody reaction using antibody or antigen that reacts specifically with labeled antigen or antibody and determining the reaction product by conventional methods when the probe is labeled by antigen or antibody. Further, detection can be performed by analyzing a chemical substance when the probe has been labeled by a chemical substance.

When soil or underground water contaminated by chlorinated ethylene or ethane is to be purified using chlorinated ethylene-decomposing bacteria, it is possible to pre-determine whether or not dechlorination can be thorough by detecting chlorinated ethylene-decomposing bacteria by the above-mentioned method. Moreover, application of measures, including addition of chlorinated ethylene-decomposing bacteria, become possible when chlorinated ethylene-decomposing bacteria have not been detected.

The method of decomposing chlorinated ethylene or ethane of the present invention is the method wherein the above-mentioned detection of ethylene-decomposing bacteria of the present invention is conducted using underground water or soil as the sample, the underground water or soil, in which chlorinated ethylene-decomposing bacteria have been detected, or cultivation liquid inoculated with these (there are cases hereafter where these are collectively referred to as chlorinated ethylene-decomposing bacteria-detected matter) is introduced to soil or underground water contaminated by chlorinated ethylene or ethane (there are cases hereafter where these are collectively referred to as contaminated environment) and the chlorinated ethylene or ethane is decomposed.

The chlorinated ethylene-decomposing bacteria-detected matter to be introduced to the contaminated environment may be any one which is detected (collected) anywhere. For instance, underground water or soil, in which chlorinated ethylene-decomposing bacteria have been detected in a place uncontaminated by chlorinated ethylene or ethane, or cultivation liquid inoculated with these, may be introduced to soil or underground water contaminated by chlorinated ethylene or ethane. Moreover, underground water or soil, in which chlorinated ethylene-decomposing bacteria have been detected in a place contaminated by chlorinated ethylene or ethane, or cultivation liquid inoculated with these, may be introduced to a place contaminated by chlorinated ethylene or ethane in the same region or may be introduced to a different place not in the same region.

The method of spreading chlorinated ethylene-decomposing bacteria-detected matter on the surface of contaminated soil, the method of injection into soil from an injection tube (injection well), the method of injection into source of underground water, are given as methods of introducing chlorinated ethylene-decomposing bacteria-detected matter into a contaminated environment. The introduction point may, of course, be the contaminated site, or upstream from the contaminated environment.

When the chlorinated ethylene or ethane is decomposed, there are cases where the underground water or soil, in which chlorinated ethylene-decomposing bacteria have been detected, or cultivation liquid inoculated with these, is simply introduced to the contaminated environment, but depending on the case, water, nutrient source, etc., may also be further introduced. Moreover, if there is not thorough decomposition with the first introduction, introduction can be repeated. It is also possible to introduce the chlorinated ethylene-decomposing bacteria-detected matter after adding coagulant to coagulate, or after supporting the matter on a carrier.

Thus, a contaminated environment contaminated by chlorinated ethylene or ethane can be purified by decomposing chlorinated ethylene or ethane.

The nucleic acid of the present invention is novel and useful. The nucleic acids of the present invention have a specific base sequence and hybridizes preferentially to 16S rRNA or rDNA of chlorinated ethylene-decomposing bacteria. Therefore, they can be used for detection of chlorinated ethylene-decomposing bacteria.

The nucleic acids for detection of chlorinated ethylene-decomposing bacteria of the present invention comprise the above-mentioned nucleic acids and therefore, chlorinated ethylene-decomposing bacteria can be detected easily with specifically high reliability by using these nucleic acids.

The labeled probes for detecting chlorinated ethylene-decomposing bacteria of the present invention label the above-mentioned nucleic acid and therefore, it is possible to easily detect with specifically high reliability chlorinated ethylene-decomposing bacteria using this label as the indicator.

The method of detecting chlorinated ethylene-decomposing bacteria of the present invention uses the above-mentioned nucleic acids or labeled probes and therefore, chlorinated ethylene-decomposing bacteria can be detected easily with specifically high reliability.

By means of the method of decomposing chlorinated ethylene or ethane of the present invention, underground water or soil, in which chlorinated ethylene-decomposing bacteria have been detected by the above-mentioned detection method, or cultivation liquid inoculated with these, is introduced to soil or underground water contaminated by chlorinated ethylene or ethane and the chlorinated ethylene or ethane is decomposed. Therefore, chlorinated ethylene or ethane can be easily and efficiently decomposed to purify the environment.

Examples of the present invention will now be described:

### EXAMPLE 1

Underground water was sampled from a total of 6 places, points A, B and C where conversion to ethylene (dechlorination) is occurring and points D, E and F where conversion to ethylene is not occurring, and DNA was extracted from 100 mL of this underground water as described below:

(1) Extraction of DNA

After filtering 100 mL underground water with a filter having a pore diameter of 0.2 μm, this filter was introduced to a tube with a capacity of 2 mL. 1 mL zirconia/silica beads (diameter of 0.1 m) and 1 ml Extraction buffer (100 mM Tris-HCl [pH 8.0], 100 mM sodium EDTA [pH 8.0], 100 mM sodium phosphate [pH 8.0], 1.5 M NaCl) were further added to this tube and treated for 2 minutes with the cell crusher Bead Beater. After repeating freezing and thawing 3 times, 10 μL proteinase K (10 mg/ml) were added and kept at a temperature of 37° C. for 30 minutes. Then 250 μL of a 10% SDS solution were added to this liquid and kept at 65° C. for 2 hours. The above-mentioned Bead Beater treatment was again performed. This was followed by centrifugation for 10 minutes at 8,000×g under room temperature. The supernatant was collected. The supernatant was extracted with chloroform. The equivalent amount of isopropanol was added and then it was set aside for 60 minutes at room temperature. Centrifugation was performed for 20 minutes at 8,000×g under room temperature and the DNA was allowed to precipitate. The precipitate was washed with 70% ethanol and then allowed to dry. Then it was dissolved in 50 μL sterile distilled water.

PCR was performed as described below using this extracted DNA solution and the presence of chlorinated ethylene-decomposing bacteria was examined.

(2) Amplification of 16S rDNA by PCR

The 16S rDNA was amplified by PCR using 1 μL of the extracted DNA solution obtained by above-mentioned (1) as the template. The total volume of the reaction solution of PCR amplification was brought to 100 μL, and 2.5 U Ex Taq DNA polymerase (Takara Shuzo) and 200 μM dNTP were used. 20 pmol each of 6 sets of primer pairs, where any one of KWI-De1 through KWI-De6 served as the upper primer and Bact1492 (5′-ACGG C/T TACCTTGTTAGGACTT-3′) served as the lower primer, and 9 sets of primer pairs, where with Bact0011 (5′-GTTTGATCCTGGCTCAG-3′) served as the upper primer and any one of complementary base sequence of KWI-De7 through KWI-De15 served as the lower primer, were used as the primer pair, as shown in Table 1. The rest of the reaction liquid composition was in accordance with the manual accompanying the PCR kit. The PCR reaction was performed by pre-heating at 94° C. for 2 minutes, followed by 30 cycles of step 1 at 94° C. for 20 seconds, step 2 at 55° C. for 30 seconds, and step 3 at 72° C. for 2 minutes, and finally, post-extension for 7 minutes at 72° C.

2 μL of the above-mentioned PCR reaction liquid were submitted to agarose electrophoresis and it was concluded that chlorinated ethylene-decomposing bacteria were present if DNA fragment of the expected size was synthesized. The results are shown in Table 1.

TABLE 1
Results of detecting chlorinated ethylene-decomposing bacteria from
underground water

| | | | Length of | | | | | | |
|---|---|---|---|---|---|---|---|---|---|
| | Upper | Lower | synthetic | Point | | | | | |
| No | Primer | Primer | DNA (kb) | A | B | C | D | E | F |
| 1 | KWI-De1 | Bact1492 | 1.38 | ◯ | X | ◯ | X | X | X |
| 2 | KWI-De2 | Bact1492 | 1.34 | ◯ | ◯ | ◯ | X | X | X |
| 3 | KWI-De3 | Bact1492 | 1.31 | ◯ | ◯ | ◯ | X | X | X |
| 4 | KWI-De4 | Bact1492 | 1.28 | ◯ | ◯ | X | X | X | X |
| 5 | KWI-De5 | Bact1492 | 1.26 | ◯ | ◯ | ◯ | X | X | X |
| 6 | KWI-De6 | Bact1492 | 1.24 | ◯ | ◯ | ◯ | X | X | X |
| | | Base se- | | | | | | | |
| | | quence | | | | | | | |
| | | complemen- | | | | | | | |
| | | tary to | | | | | | | |
| 7 | Bact0011 | KWI-De7 | 0.91 | X | ◯ | ◯ | X | X | X |
| 8 | Bact0011 | KWI-De8 | 0.82 | ◯ | ◯ | ◯ | X | X | X |
| 9 | Bact0011 | KWI-De9 | 0.80 | ◯ | ◯ | ◯ | X | X | X |
| 10 | Bact0011 | KWI-De10 | 0.98 | ◯ | X | ◯ | X | X | X |
| 11 | Bact0011 | KWI-De11 | 1.01 | ◯ | ◯ | ◯ | X | X | X |
| 12 | Bact0011 | KWI-De12 | 1.10 | ◯ | ◯ | ◯ | X | X | X |
| 13 | Bact0011 | KWI-De13 | 1.22 | ◯ | ◯ | ◯ | X | X | X |
| 14 | Bact0011 | KWI-De14 | 1.24 | ◯ | ◯ | X | X | X | X |
| 15 | Bact0011 | KWI-De15 | 1.40 | ◯ | ◯ | ◯ | X | X | X |

◯: Synthesis of DNA observed
X: Synthesis of DNA not observed

The base sequence of KWI-De1˜KWI-De15 in Table 1 are as shown in Table 2.

TABLE 2

| | |
|---|---|
| | Base sequence |
| Sequence No. | (from 5′ to 3′) |

| | | |
|---|---|---|
| KWI-De1 | Sequence No. | GTCTTAAGCAATTAAGATAG |
| | 1 | |
| KWI-De2 | Sequence No. | CGCGTAAGTAACCTACCTCTAAGT |
| | 2 | |
| KWI-De3 | Sequence No. | GCTTCGGGAAACTGAAGG |
| | 3 | |
| KWI-De4 ^{*1} | Sequence No. | TGGRCCGACATATGTTGGTT |
| | 4 | |
| KWI-De5 | Sequence No. | CACTAAAGCCGTAAGGCGCT |
| | 5 | |
| KWI-De6 | Sequence No. | TGGTGAGGGGCTTGCGTCCG |
| | 6 | |
| KWI-De7 | Sequence No. | GTGAGCGTAGGTGGTCTTTC |
| | 7 | |
| KWI-De8 | Sequence No. | GAGCAGGAGAAAACGGAATT |
| | 8 | |
| KWI-De9 | Sequence No. | GTATAGGGAGTATCGACCC |
| | 9 | |
| KWI-De10 | Sequence No. | TGTAGTAGTGAACTGAAAGGGGAAC |
| | 10 | |
| KWI-De11 | Sequence No. | GACCTGTTAAGTCAGGAACTTGCAC |
| | 11 | |
| KWI-De12 | Sequence No. | TGTTGCTAGTTAAATTTTC |
| | 12 | |
| KWI-De13 | Sequence No. | GTTGCAACAGTGCGAACTGG |
| | 13 | |
| KWI-De14 | Sequence No. | GCTAATCCCCAAAGCTGTC |
| | 14 | |
| KWI-De15 | Sequence No. | GTCGATGTGCCAACCGCAAGG |
| | 15 | |

^{*1} The R in the base sequence is A or G.

Based on the results in Table 1, DNA synthesis was observed in all but 5 of 45 times by the above-mentioned PCR and electrophoresis at points A, B and C, where ethylene conversion is occurring. On the other hand, no DNA synthesis whatsoever was observed at points D, E and F, where no ethylene conversion whatsoever is occurring. Based on these results, chlorinated ethylene-decomposing bacteria are always present and they can be monitored at points where ethylene conversion is occurring.

### EXAMPLE 2

(1) Detection by Light Cycler

PCR detection of even higher reliability was conducted on the extracted DNA solutions of Example 1 using the Light Cycler made by Roche Diagnostics Co., Ltd.

In this case, KWI-De8 was used as the upper primer and oligonucleotide complementary to KWI-De15 was used as the lower primer. Moreover, KWI-De10 labeled with FITC (fluorescence isothiocyanate) at the 3′ terminal and KWI-De11 phosphorylated at the 3′ terminal and labeled with FITC at the 5′ terminal were used as the hybridization probe. The PCR reaction was carried out using Light Cycler DNA Master Hybridization Probes Kit (Trademark) in accordance with the manual thereof. The reaction conditions are shown in Tables 3˜6.

TABLE 3
Denaturation

Number of cycles = 1

| | Target | Storage | Speed of temp. | Fluorescence |
|---|---|---|---|---|
| Segment | temperature (° C.) | time (s) | change (° C./s) | detected |
| 1 | 95 | 120 | 20 | None |

TABLE 4
Denaturation

Number of cycles = 50 (segment 1 → 2 → 3 back to 1)

| | Target | Storage | Speed of temp. | Fluorescence |
|---|---|---|---|---|
| Segment | temperature (° C.) | time (s) | change (° C./s) | detected |
| 1 | 95 | 0 | 20 | None |
| 2 | 54 | 15 | 20 | Detected once |
| 3 | 72 | 30 | 2 | None |

TABLE 5
Denaturation

Number of cycles = 1

| | Target | Storage | Speed of temp. | Fluorescence |
|---|---|---|---|---|
| Segment | temperature (° C.) | time (s) | change (°C./s) | detected |
| 1 | 95 | 0 | 20 | None |
| 2 | 44 | 10 | 20 | None |
| 3 | 85 | 0 | 0.2 | Continuously |
| | | | | detected |

TABLE 6
Denaturation

Number of cycles = 1

| | Target | Storage | Speed of temp. | Fluorescence |
|---|---|---|---|---|
| Segment | temperature (° C.) | time (s) | change (° C./s) | detected |
| 1 | 40 | 30 | 20 | None |

The results showed that the desired DNA can be synthesized by PCR and therefore, chlorinated ethylene-decomposing bacteria are present in the underground water at points A, B and C. Nevertheless, it was concluded that chlorinated ethylene-decomposing bacteria are not present at points D, E and F because the desired DNA was not synthesized. Thus, it is clear that the primers in Table 2 can be used as the hybridization probe as well.

### EXAMPLE 3

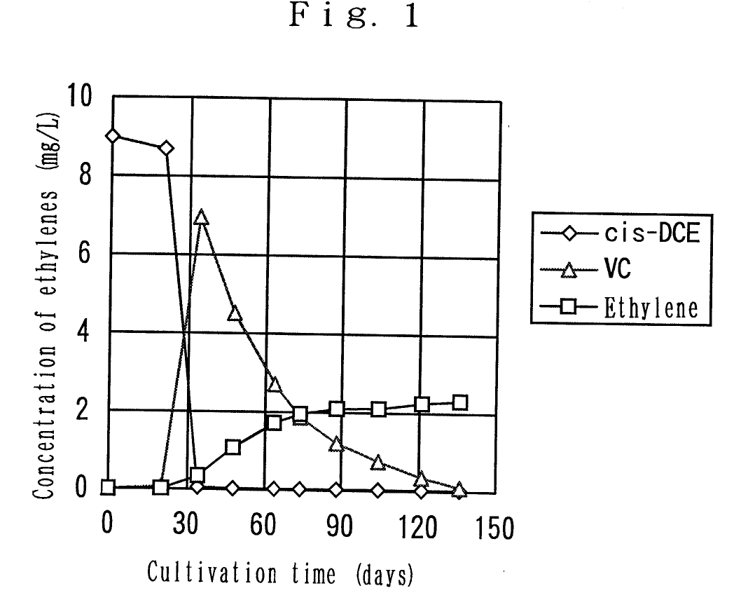

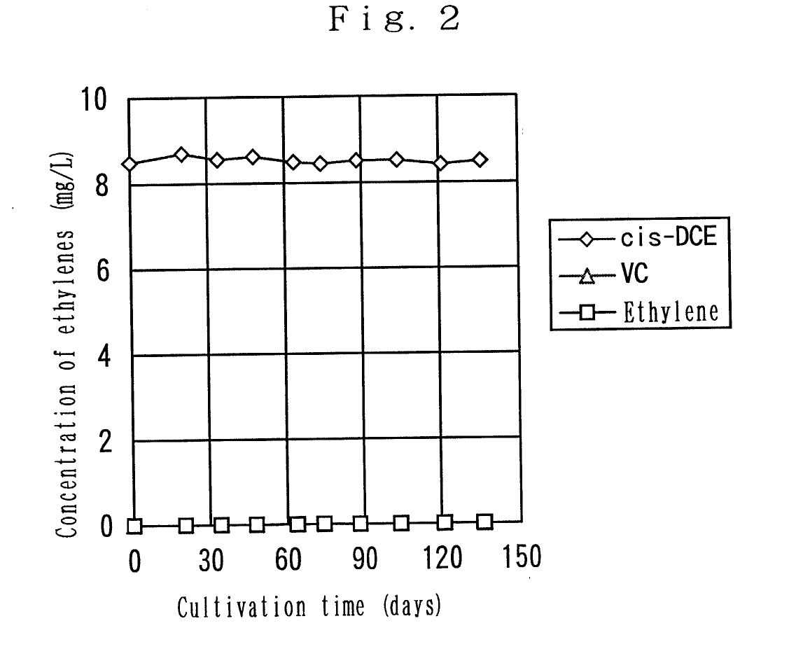

100 g soil and 50 mL underground water contaminated by cis-dichloroethylene (cis-DCE) were introduced to a vial with a capacity of 150 mL. Lactic acid was added to a concentration of 100 mg/L and then the bottle was closed with a butyl rubber stopper and sealed with an aluminum cap. Two of these same vials were used. Bacterium suspension in which chlorinated ethylene-decomposing bacterium genes had been detected were transferred in one vial to a final concentration of chlorinated ethylene-decomposing bacterium genes of 10⁵ copies/mL. Moreover, genes were not transferred to the other vial, which served as the control. These vials were set aside at 30° C. for cultivation. They were periodically sampled and the concentration of ethylenes in the vials were determined. The results are shown in FIGS. 1 and 2.

There was marked chlorinated ethylene decomposition and vinyl chloride (VC) was first detected approximately 20 days after starting the experiment when bacterium suspension in which chlorinated ethylene-decomposing bacterium genes had been detected was added (See FIG. 1). Thereafter, the VC was also decomposed, with there being complete conversion to ethylene in approximately 135 days. On the other hand, no vinyl chloride or ethylene whatsoever was detected throughout the experimental period in the case of the control (See FIG. 2).

Based on the above-mentioned results, it is clear that adding a liquid in which chlorinated ethylene-decomposing bacteria have been detected has the effect of promoting the chlorinated ethylene-decomposition reaction.

### EXAMPLE 4

Wells (A and B) were placed in 2 places that were 1 m apart in an area contaminated by chlorinated ethylene. Water was pumped from point B at 3 L/min and this water was introduced to point A. When introduced to point A, lactic acid was added at a concentration of 100 mg/L. The contaminated aquifer was 3 m below the ground and the aquifer was 4 m thick.

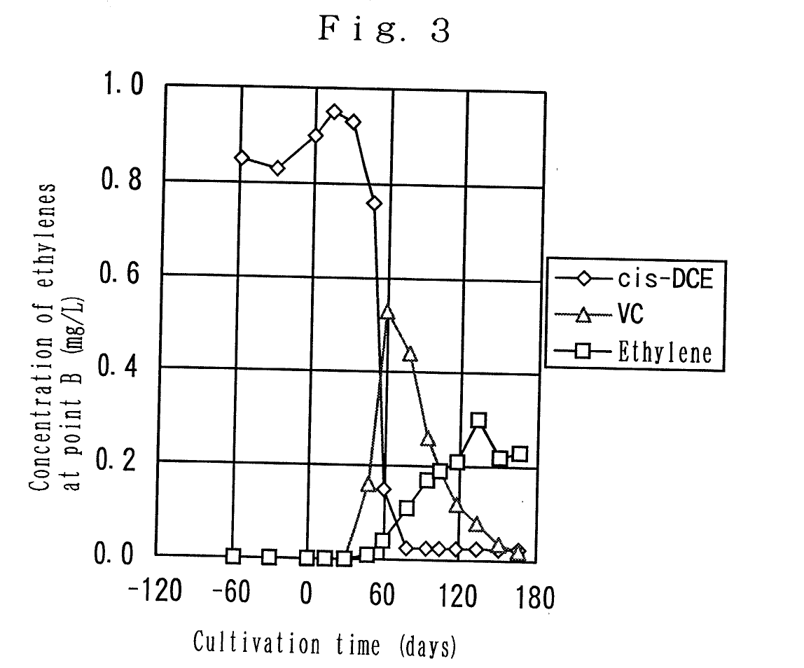

The underground water was periodically sampled at point B and the concentration of ethylenes was determined. The results are shown in FIG. 3. The axis of abscissas shows the time that had lapsed and the 0 point is the time when 50 L of liquid in which chlorinated ethylene-decomposing bacteria had been detected (gene concentration: 10⁷ copies/mL) had been introduced from point A.

As is clear from the results in FIG. 3, no decomposition of dichloroethylene whatsoever was seen prior to introduction, but there was marked decomposition 20 days after introduction, with conversion to ethylene being 100% after approximately 170 days.

Based on the above-mentioned results, it is clear that adding liquid in which chlorinated ethylene-decomposing bacteria have been detected has the effect of promoting the chlorinated ethylene decomposition reaction, even in areas contaminated by chlorinated ethylene.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

What is claimed is:

1. Nucleic acid, comprising 18˜25 nucleotides, which preferentially hybridizes to at least one of the 16S rRNA and the rDNA of chlorinated ethylene-decomposing bacteria and has a base sequence selected from the group consisting of (a) a base sequence of SEQ ID No. 1 through No. 15, (b) a base sequence having at least 90% homology with a base sequence of SEQ ID No. 1 through No. 15, and (c) a base sequence complementary to said base sequence of one of (a) and (b).

2. Nucleic acid, comprising 10˜50 nucleotides, which preferentially hybridizes to at least one of the 16S rRNA and the rDNA of chlorinated ethylene-decomposing bacteria, wherein a base sequence of at least 10 individual bases in succession has a base sequence selected from the group consisting of (d) the same as any of base sequences of SEQ ID No. 1 through No. 15, and (e) a base sequence complementary to said base sequence of (d).

3. A labeled probe for the detection of chlorinated ethylene-decomposing bacteria comprising said nucleic acid of claim 1 which is labeled by at least one of a radioactive element, enzyme, fluorescent substance, antigen, antibody, and chemical substance.

4. A labeled probe for the detection of chlorinated ethylene-decomposing bacteria comprising said nucleic acid of claim 2 which is labeled by at least one of a radioactive element, enzyme, fluorescent substance, antigen, antibody, and chemical substance.

5. A method of detecting chlorinated ethylene-decomposing bacteria in a sample, comprising:
performing PCR (polymerase chain reaction) using said nucleic acid of claim 1 as the primer and a nucleic acid in said sample as template; and
detecting the DNA fragment that has been synthesized.

6. A method of detecting chlorinated ethylene-decomposing bacteria in a sample, comprising:
performing PCR (polymerase chain reaction) using said nucleic acid of claim 2 as the primer and a nucleic acid in said sample as template; and
detecting the DNA fragment that has been synthesized.

7. A method of detecting chlorinated ethylene-decomposing bacteria, comprising:
bringing said labeled probe for detecting chlorinated ethylene-decomposing bacteria of claim 3 into contact with one of a sample and nucleic acid prepared from a sample to perform RNA or DNA hybridization; and
detecting chlorinated ethylene-decomposing bacteria using the label as an indicator.

8. A method of detecting chlorinated ethylene-decomposing bacteria, comprising:
bringing said labeled probe for detecting chlorinated ethylene-decomposing bacteria of claim 4 into contact with one of a sample and nucleic acid prepared from a sample to perform RNA or DNA hybridization; and
detecting chlorinated ethylene-decomposing bacteria using the label as an indicator.

9. A method of decomposing at least one of chlorinated ethylene and chlorinated ethane in a substance to be treated, comprising:
performing said detection of chlorinated ethylene-decomposing bacteria of claim 5; and
introducing a material, in which chlorinated ethylene-decomposing bacteria have been detected, or a cultivation liquid inoculated with said chlorinated ethylene-decomposing bacteria, to said substance contaminated by chlorinated ethylene or chlorinated ethane.

10. The method of decomposing at least one of chlorinated ethylene and chlorinated ethane in a substance to be treated wherein said substance is at least one of underground water and soil.

11. A method of decomposing at least one of chlorinated ethylene and chlorinated ethane in a substance to be treated, comprising:
performing said detection of chlorinated ethylene-decomposing bacteria of claim 6; and
introducing a material, in which chlorinated ethylene-decomposing bacteria have been detected, or a cultivation liquid inoculated with said chlorinated ethylene-decomposing bacteria, to said substance contaminated by chlorinated ethylene or chlorinated ethane.

12. The method of decomposing at least one of chlorinated ethylene and chlorinated ethane in a substance to be treated according to claim 11 wherein said substance is one of underground water and soil.

13. A method of decomposing at least one of chlorinated ethylene and chlorinated ethane in a substance to be treated, comprising:
performing said detection of chlorinated ethylene-decomposing bacteria of claim 7; and
introducing a material, in which chlorinated ethylene-decomposing bacteria have been detected, or a cultivation liquid inoculated with said chlorinated ethylene-decomposing bacteria, to said substance contaminated by chlorinated ethylene or chlorinated ethane.

14. The method of decomposing at least one of chlorinated ethylene and chlorinated ethane in a substance to be treated according to claim 13 wherein said substance is one of underground water and soil.

15. A method of decomposing at least one of chlorinated ethylene and chlorinated ethane in a substance to be treated, comprising:
performing said detection of chlorinated ethylene-decomposing bacteria of claim 8; and
introducing a material, in which chlorinated ethylene-decomposing bacteria have been detected, or a cultivation liquid inoculated with said chlorinated ethylene-decomposing bacteria, to said substance contaminated by chlorinated ethylene or chlorinated ethane.

16. The method of decomposing at least one of chlorinated ethylene and chlorinated ethane in a substance to be treated according to claim 15 wherein said substance is one of underground water and soil.

17. A method of decomposing at least one of chlorinated ethylene and chlorinated ethane in a substance to be treated, comprising:
detecting chlorinated ethylene-decomposing bacteria in a material by performing PCR (polymerase chain reaction) using a first nucleic acid as the primer and a nucleic acid in said detection sample as template, and detecting the DNA fragment that has been synthesized;
said first nucleic acid comprising 18˜25 nucleotides which preferentially hybridizes to at least one of the 16S rRNA and the rDNA of chlorinated ethylene-decomposing bacteria and has a base sequence selected from the group consisting of (a) a base sequence of SEQ ID No. 1 through No. 15, (b) a base sequence having at least 90% homology with abase sequence of SEQ ID No. 1 through No. 15, and (c) a base sequence complementary to said base sequence of one of (a) and (b); and
introducing said material, in which chlorinated ethylene-decomposing bacteria have been detected, or a cultivation liquid inoculated with said chlorinated ethylene-decomposing bacteria, to said substance contaminated by chlorinated ethylene or chlorinated ethane.

18. The method of decomposing at least one of chlorinated ethylene and chlorinated ethane in a substance to be treated according to claim 17 wherein said substance is one of underground water and soil.

19. A method of decomposing at least one of chlorinated ethylene and chlorinated ethane in a substance to be treated, comprising:
providing a labeled probe for the detection of chlorinated ethylene-decomposing bacteria having a nucleic acid, comprising 18˜25 nucleotides which preferentially hybridizes to at least one of the 16S rRNA and the rDNA of chlorinated ethylene-decomposing bacteria and has a base sequence selected from the group consisting of (a) a base sequence of SEQ ID No. 1 through No. 15, (b) a base sequence having at least 90% homology with a base sequence of SEQ ID No. 1 through No. 15, and (c) a base sequence complementary to said base sequence of one of (a) and (b), which is labeled by at least one of a radioactive element, enzyme, fluorescent substance, antigen, antibody, and chemical substance;
bringing said labeled probe for detecting chlorinated ethylene-decomposing bacteria into contact with a material to perform RNA or DNA hybridization;
detecting chlorinated ethylene-decomposing bacteria using the label as an indicator; and
introducing said material, in which chlorinated ethylene-decomposing bacteria have been detected, or a cultivation liquid inoculated with said chlorinated ethylene-decomposing bacteria, to said substance contaminated by chlorinated ethylene or chlorinated ethane.

20. The method of decomposing at least one of chlorinated ethylene and chlorinated ethane in a substance to be treated according to claim 19 wherein said substance is one of underground water and soil.
